(19) United States
(12) Patent Application Publication
(10) Pub. No.: US 2010/0221835 A1
(43) Pub. Date: Sep. 2, 2010

(54) METHOD FOR CARTILAGE TISSUE REGENERATION VIA SIMULATED MICROGRAVITY CULTURE USING SCAFFOLDS

(75) Inventors: Junzo Tanaka, Ibaraki (JP); Yoshito Ikada, Ibaraki (JP); Yoshimi Ohyabu, Ibaraki (JP); Toshimasa Uemura, Ibaraki (JP)

Correspondence Address: SUGHRUE MION, PLLC, 2100 PENNSYLVANIA AVENUE, N.W., SUITE 800, WASHINGTON, DC, 20037, US

(73) Assignee: NATIONAL INSTITUTE OF ADVANCED INDUSTRIAL SCIENCE AND TECHNOLOGY, Chiyoda-ku, Tokyo (JP)

(21) Appl. No.: 11/917,226

(22) PCT Filed: Jun. 15, 2006
(86) PCT No.: PCT/JP2006/312457

§ 371 Date: Dec. 12, 2007

(30) Foreign Application Priority Data
Jun. 15, 2005 (JP) 2005-174932

(51) Int. Cl. C12N 5/077

(52) U.S. Cl. 435/396; 435/395

(57) ABSTRACT

This invention relates to a method for cartilage tissue engineering using scaffolds in simulated microgravity culture. This invention enables engineering of homogeneous cartilage tissue using bone marrow cells in a more rapid manner in a simulated microgravity environment, while allowing control of the configuration of the resulting cartilage tissue.

### TECHNICAL FIELD

The present invention relates to a method for cartilage tissue engineering via simulated microgravity culture using scaffolds. More particularly, the present invention relates to a method for cartilage tissue engineering by seeding bone marrow cells on collagen-based scaffolds or the like and culturing the composite in a simulated microgravity environment.

### BACKGROUND ART

Three-dimensional tissue regeneration from cells generally requires three-dimensional culture or stirred culture with appropriate scaffolds. With conventional techniques, however, considerable mechanical stimuli and damages are imposed on cells. Accordingly, it is difficult to obtain a large tissue mass. Even if a large tissue mass were to be obtained, the inner region of the formed tissue would be likely to become necrotic.

In order to overcome such drawbacks, there are sets of bioreactors designed to optimize gravity. For example, the rotating wall vessel (RWV) bioreactor is a NASA-developed rotary bioreactor equipped with gas exchange membranes. The RWV bioreactor, which is a uniaxial, rotary, horizontal, and cylindrical bioreactor, is filled with a culture medium, the cells are seeded therein, and the bioreactor rotates along the horizontal axis of the cylinder to culture the cells. Because of the stress resulting from rotation, a microgravity environment is realized in the bioreactor, which provides gravity that is approximately 1/100 of the ground gravity. Accordingly, cells can grow while being homogeneously suspended in a culture medium, and they aggregate to form a large tissue mass. Some rotary bioreactors, such as a biaxial clinostat, rotate in multiaxial directions. Since multiaxial bioreactors cannot minimize shear stress, it is difficult to realize an ideal simulated microgravity environment.

The inventors have already reported that use of RWV would realize the regeneration of three-dimensional cartilage tissue from bone-marrow-derived mesenchymal stem cells without the use of special cellular scaffolds (Abstracts of the Annual Meeting of the Japanese Biomaterials Society, 2003, p. 271). With this technique, however, the shape of the constructed cartilage tissue cannot be controlled. This disadvantageously limits the clinical applications thereof, where engineering of tissue that is suitable for the damaged area is required.

To date, RWV-based cell culture has been attempted using a variety of cells, and the feasibility of cartilage tissue engineering via construction of a composite of scaffolds (PLGA) and cartilage cells has been verified (Freed, L E, Hollander, A P, Martin, I, Barry, J R, Langer, R, and Vunjak-Novakovic, G, Chondrogenesis in a cell-polymer-bioreactor system, Exp. Cell Res., Apr. 10, 1998, 240(1), 58-65). However, there is no report concerning the engineering of cartilage tissue from bone marrow cells in RWV rotation culture using scaffolds.

A variety of cellular scaffolds for static culture have been known; however, types of scaffolds that are suitable for cell culture in a simulated microgravity environment in a rotary bioreactor cannot be predicted based on the results of static culture.

### DISCLOSURE OF THE INVENTION

An object of the present invention is to provide a method for engineering of homogeneous cartilage tissue from bone marrow cells in a more rapid manner in a simulated microgravity environment, while allowing control of the shape of the formed cartilage tissue.

The present inventors have attempted cartilage tissue regeneration from bone marrow cells in a simulated microgravity environment by a rotating-wall vessel (RWV) bioreactor using a variety of scaffolds. As a result, they discovered that cartilage tissue of high quality could be engineered only when collagen sponges were used as cellular scaffolds.

Specifically, the present invention relates to a method for engineering cartilage tissue by seeding bone marrow cells on scaffolds and culturing the composite in a simulated microgravity environment.

Examples of scaffolds that can be preferably used include collagen-based scaffolds and polymer-based scaffolds such as polycaprolactone or polyglycolic acid.

In this method, the gravity is preferably approximately 1/10 to 1/100 of the ground gravity on a time-average basis in a simulated microgravity environment. Such simulated microgravity environment can be attained with the use of a bioreactor that realizes the simulated microgravity environment on the ground by compensating for ground gravity by the mechanical stress originated from rotation.

A uniaxial rotary bioreactor is preferably used, and an example thereof is the rotating wall vessel (RWV) bioreactor. When the RWV bioreactor is used, culture is preferably conducted at a seeding density of 10⁶ to 10⁷ cells/cm³ at a rotation speed of approximately 8.5 to 25 rpm (in a 5-cm vessel), for example. It should be noted that the culture conditions are not limited thereto.

In the method of the present invention, it is preferable that an inducer of cartilage differentiation, such as TGF-β or dexamethasone, be added to a culture medium.

The scaffolds according to the present invention preferably have spongy structures. When collagen-based scaffolds are used, use of type I or II collagen is preferable. When polymer-based scaffolds are used, use of polycaprolactone-based or polyglycolic-acid-based scaffolds is preferable.

According to an embodiment of the present invention, bone marrow cells isolated from a target (patient) in need of cartilage tissue transplantation are used. Cartilage tissue engineered from bone marrow cells isolated from the target of transplantation is free from the risk of rejection or the like by the patient. Thus, such tissue can be preferably used to regenerate and/or repair cartilage defects of the target.

According to the present invention, homogeneous cartilage tissue of a desired shape can be more rapidly engineered from bone marrow cells. Accordingly, the present invention is highly applicable in clinical settings, such as regenerative medicine aimed at treating rheumatoid arthritis or osteoarthritis in the orthopedic field or at repairing auricular cartilage in the plastic surgery field.

### BRIEF DESCRIPTION OF THE DRAWINGS

FIG. 1 shows the experimental protocol for Example 1.

FIG. 2 shows stained images of the cartilage tissue cultured in an RWV bioreactor for 2 weeks: (A) hematoxylin-eosin staining; (B) safranin O staining; and (C) toluidine blue staining.

FIG. 3 shows a chart representing a GAG content of cartilage tissue engineered in an RWV bioreactor (diagonal line: when collagen sponges were used; outlined: when cells were seeded in a culture medium (the control); *: significant difference of p<0.05).

FIG. 4 shows a chart representing the compression strength of the cartilage tissue engineered in an RWV bioreactor (diagonal line: when collagen sponges were used; outlined: when cells were seeded in a culture medium (the control); *: significant difference of p<0.05).

FIG. 5 shows the results of immunostaining of cartilage tissue engineered via culture in an RWV bioreactor for 2 weeks with the use of collagen sponges with the anti-type I collagen antibody (A) and with the anti-type II collagen (B).

FIG. 6 shows (A) an macroscopic appearance and (B) a safranin O stained image of the engineered cartilage tissue cultured in an RWV bioreactor for 2 weeks after seeding cells in a culture medium.

FIG. 7 shows the phase contrast-fluorescent (DAPI) microscopic image after static culture (A) and after RWV rotation culture (B) of the cells/collagen sponge composites for 2 weeks.

FIG. 8 shows the toluidine blue-stained image (×40) (A) and the SEM image of the cells/OPLA composite (×300) (B) after 2 weeks culture.

FIG. 9 shows the toluidine blue-stained image (×40) (A), the SEM image of he cells/HAP-HA composite (×200) (B), and the phase-contrast microscopic image of the composite (×40) (C) after 2-week culture

FIG. 10 shows the immunostaining results using anti-type I collagen antibody for evaluating the influence of scaffolds (collagen sponges) on cartilage tissue regeneration using an RWV bioreactor:

without collagen sponges: (A) 2 weeks after transplantation, (B) 4 weeks after transplantation;

with collagen sponges: (C) 2 weeks after transplantation, (D) 4 weeks after transplantation.

FIG. 11 shows the immunostaining results using anti-type II collagen antibody for evaluating the influence of scaffolds (collagen sponges) on cartilage tissue regeneration:

without collagen sponges: (A) 2 weeks after transplantation, (B) 4 weeks after transplantation;

with collagen sponges: (C) 2 weeks after transplantation, (D) 4 weeks after transplantation.

FIG. 12 shows the immunostaining results using anti-proteoglycan antibody: for evaluating the influence of scaffolds (collagen sponges) on cartilage tissue regeneration:

without collagen sponges: (A) 2 weeks after transplantation, (B) 4 weeks after transplantation;

with collagen sponges: (C) 2 weeks after transplantation, (D) 4 weeks after transplantation.

This description includes part or all of the contents as disclosed in the description of Japanese Patent Application No. 2005-174932, which is a priority document of the present application.

### PREFERRED EMBODIMENTS OF THE INVENTION

Hereafter, the present invention is described in detail.

### 1. Simulated Micro Gravity Environment

In the present invention, the term “simulated microgravity environment” refers to a simulated microgravity environment that imitates the microgravity environment found in space. Such simulated microgravity environment is realized by compensating the ground gravity by mechanical stress originating from rotation, for example. More specifically, a rotating substance receives a force that is represented by the vector sum of the ground gravity and the mechanical stress, and thus, the magnitude and the direction thereof vary depending on time. That is, a force that is excessively smaller than the ground gravity (g) acts on a substance on a time-average basis. This allows realization of a “simulated microgravity environment” that is very similar to that in the space.

In a “simulated microgravity environment,” it is necessary that cells grow, differentiate, and are homogeneously dispersed without sinking and that they three-dimensionally aggregate to form tissue masses. In other words, the rotation speed is preferably adjusted to synchronize with the sinking speed of the seeded cells to minimize the influence of the ground gravity on the cells. More specifically, the microgravity applied to the cultured cells is preferably approximately 1/10 to 1/100 of the ground gravity (g) on a time-average basis.

### 2. Bioreactor

In the present invention, a rotary bioreactor is used in order to realize a simulated microgravity environment. Examples of bioreactors that can be used include the Rotating-Wall Vessel (RWV, U.S. Pat. No. 5,002,890), the Rotary Cell Culture System™ (RCCS, Synthecon Incorporated), a 3D-clinostat, and bioreactors disclosed in JP Patent Publication (Unexamined) Nos. 8-173143 (1996), 9-37767 (1997), and 2002-45173. Some of these bioreactors are uniaxial rotary bioreactors and others are at least biaxial rotary bioreactors. In the present invention, use of a uniaxial rotary bioreactor is preferable. When a multiaxial bioreactor (such as a biaxial clinostat) is used, shear stress cannot be minimized, and the sample itself rotates. Thus, a sample cannot be maintained at a stationary position in a vessel, as in a case involving the use of a uniaxial reactor. This stationary state is an important condition for attaining a large three-dimensional tissue mass without the use of specific scaffolds. RWV and RCCS are particularly preferable because they are equipped with gas exchange membranes.

The RWV that is used in the examples of the present invention is a NASA-developed uniaxial rotary bioreactor equipped with gas exchange membranes. The RWV bioreactor, which is a horizontal cylindrical bioreactor, is filled with a culture medium, the cells are seeded therein, and the bioreactor rotates along the horizontal axis of the cylinder to conduct culture. The “microgravity environment” that provides considerably lower gravity than the ground gravity is virtually realized in the bioreactor because of the stress by rotation. In such simulated microgravity environment, cells are homogeneously suspended in a culture medium, they are cultured and grown under the minimal shear stress for a necessary period of time, and they aggregate to form tissue masses.

A preferable rotation speed when an RWV is used is adequately determined in accordance with the diameter of the vessel and the size or mass of the tissue mass. When a 5-cm vessel is used, for example, the rotation speed is preferably between about 8.5 rpm and 25 rpm. When culture is conducted at such rotation speed, the gravity acting on the cells in the vessel is substantially about 1/10 to 1/100 of the ground gravity (1 g).

### 3. Bone Marrow Cells

In the present invention, bone marrow cells are used as cell sources for cartilage tissue engineering. The bone marrow cells used in the present invention are bone-marrow-derived undifferentiated cells that are capable of differentiation and growth. Bone-marrow-derived mesenchymal stem cells are particularly preferable. In addition to the established cell lines, bone marrow cells isolated from targets (patients) in need of cartilage tissue transplantation are preferably used. Such cells are preferably prepared by isolating the bone marrow cells from the target of transplantation and removing connective tissue and the like therefrom in accordance with a conventional technique. Alternatively, primary culture may be conducted in accordance with a conventional technique and cells may be grown in advance. Further, the cultured cells isolated from the target of transplantation may be cryopreserved. Specifically, the bone marrow cells that have been isolated in advance may be cryopreserved and then used according to need.

### 4. Scaffolds

In the present invention, cells are cultured on adequate scaffolds. Scaffolds that are used herein are not particularly limited, provided that such cells are known in the art. Examples thereof include collagen-based scaffolds, polymer-based scaffolds such as polycaprolactone or polyglycolic acid-based scaffolds, and composites thereof.

When RWV rotation culture is performed, scaffolds are influenced by the flow resulting from rotation. Accordingly, scaffolds preferably have sufficient strength to not be deformed by rotation. Also, scaffolds sufficiently adhere to cells, so that the adhered cells do not remove during rotation.

The term “collagen-based cellular scaffolds” used herein refers to scaffolds that are mainly composed of collagen. Collagen sufficiently adheres to cells, and the mechanical strength thereof can be adjusted to a desired level by crosslinking. Thus, such cellular scaffolds are preferable for RWV rotation culture.

Type I collagen, which is a major component of bone and tooth organic matrix with high bioaffinity, or type II collagen, which is a major component of a cartilage matrix, are preferably used. Such type I and type II collagen may be commercially available collagens or may be extracted and purified from adequate source (e.g., animal connective tissue including porcine or bovine skin in the case of type I collagen) in accordance with a conventional technique. Alternatively, purified collagen can be lyophilized, dissolved in an acetic acid solution, and incubated with the addition of NaCl, NaOH, HEPES, or the like, in order to produce reconstituted collagen fibers.

In the present invention, collagen having a spongy structure formed by lyophilization is preferably used. Such spongy structure adds physical properties to collagen that are required for scaffolds for cell culture.

When a spongy structure is produced, lyophilization conditions (e.g., temperature, duration of lyophilization, or lyophilization in water) can be adequately regulated in accordance with, for example, the structure of a desired scaffold, i.e., specific surface area, porosity, pore size, or the like. The resulting lyophilized product can be molded according to need. The term “spongy structure” used herein refers to a flexible microporous structure (i.e., a structure comprising numerous pores (gaps) of about several μm to several tens of μm). In the present invention, the porosity of a spongy structure is preferably between 40% and 90%, and more preferably between 60% and 90%. This is because cell invasion becomes insufficient and cell strength is lowered when the porosity is outside from such range.

Collagen fibers may be crosslinked according to need. Crosslinking may involve any portions of collagens. Particularly preferable crosslinking occurs between a carboxyl group and a hydroxyl group, between a carboxyl group and a ε-amino group, or between ε-amino groups. Any method, such as chemical crosslinking using a crosslinking agent or condensing agent or physical crosslinking using γ rays, ultraviolet rays, thermal dehydration, an electron beam, or the like, may be employed. Chemical crosslinking using a crosslinking agent is particularly preferable from the viewpoint of controllability of the degree of crosslinking or biocompatibility of the resulting crosslinking product. Examples of crosslinking agents that can be used include: aldehyde crosslinking agents such as glutaraldehyde or formaldehyde; isocyanate crosslinking agents such as hexamethylene diisocyanate; carbodiimide crosslinking agents such as 1-ethyl-3-(3-dimethylaminopropyl) carbodiimide hydrochloride; polyepoxy crosslinking agents such as ethylene glycol diethyl ether; and transglutaminase. The amount of crosslinking agent used is preferably between about 10 μmol and 10 mmol per g of collagen.

Examples of “polymer-based scaffolds” used in the present invention include scaffolds composed of polymers such as polylactic acid, polyglycolic acid, polycaprolactone, D,D-L,L-polylactic acid, and hyaluronic acid. Polycaprolactone (the Abstracts of the 6th Annual Meeting of the Japanese Society for Tissue Engineering, p. 79, June 2003, Shinichi Terada et al., Induction of auricular cartilage tissue using long term biodegradable polymer) and polyglycolic acid are particularly preferable for RWV rotation culture from the viewpoint of sufficient adhesiveness to cells and adequate mechanical strength.

The scaffolds may further comprise drugs capable of accelerating cartilage cell differentiation described below or other porous hard materials such as hydroxyapatite, βTCP, or αTCP within the scope of the present invention.

### 5. Cell Culture Conditions

Culture media that are usually employed for culture of bone marrow cells, such as MEM, α-MEM, and DMEM, can be adequately selected in accordance with cell type and used for cell differentiation and proliferation. Such media may additionally contain, for example, FBS (Sigma) or antibiotics such as Antibiotic-Antimycotic (Gibco BRL).

Culture media may further contain at least one member selected from the group consisting of dexamethasone, immunosuppressants such as FK-506 or ciclosporin, bone morphogenetic proteins (BMP) such as BMP-2, BMP-4, BMP-5, BMP-6, BMP-7, or BMP-9, and osteogenic humoral factors such as TGF-β capable of accelerating cartilage cell differentiation in combination with a phosphagen such as glycerol phosphate or ascorbic acid phosphate. Addition of either or both TGF-β and dexamethasone in combination with an adequate phosphagen is particularly preferable. In such a case, the amount of TGF-β added is approximately between 1 ng/ml and 10 ng/ml, and that of dexamethasone added is up to a maximum of 100 nM. Instead of the addition of a growth factor such as TGF-β, platelet rich plasma (PRP) containing growth factors such as TGF-β can be added. The addition of platelet rich plasma (PRP) can be preferable from the viewpoint of a rejection-free safe method in clinical application.

Cell culture is preferably conducted in the presence of 3% to 10% CO₂ at 30° C. to 40° C., and particularly preferably in the presence of 5% CO₂ at 37° C. The duration of culture is not particularly limited, and it is at least 4 days, and preferably between 7 and 28 days.

When an RWV (in a 5-cm vessel) is used, bone marrow cells are seeded on collagen-based scaffolds at a seeding density of 10⁶ to 10⁷ cells/cm³, and culture is conducted using the aforementioned culture medium at a rotation speed of 8.5 to 25 rpm (in a 5-cm vessel). Under such conditions, the sinking speed of the seeded cells synchronizes with the rotation speed of the vessel, and the influence of the ground gravity imposed on the cells is minimized.

When scaffolds are not used, cells that have been cultured to overconfluence are seeded and a large cartilage tissue mass can then be obtained. When collagen scaffolds are used, however, a cartilage tissue mass can be obtained without culture to overconfluence. Specifically, the use of scaffolds can shorten the in vitro culture duration by 2 to 3 weeks (i.e., shortening of monolayer culture by a week and shortening of induction of differentiation by 1 to 2 weeks), which is a very desirable effect from the viewpoint of clinical application (i.e., transplantation).

### 6. Applications of the Invention

The application of the method according to the present invention to regenerative medicine enables cartilage tissue regeneration with the use of autologous bone marrow cells. Specifically, bone marrow cells isolated from a target (patient) in need of cartilage tissue transplantation are seeded on collagen-based scaffolds and three-dimensionally cultured in a simulated microgravity environment to engineer cartilage tissue of a desired shape, and the engineered cartilage tissue is applied to a cartilage defect of the target of transplantation. The engineered cartilage tissue is free from the risk of rejection, the level of damage imposed on normal tissue resulting from the use of the engineered cartilage tissue is lower than that resulting from the use of autologous cartilage, and a greater number of cartilage cells are obtained by culture.

This enables repair of a greater range of cartilage defects and enables safer cartilage regeneration. Accordingly, the method of the present invention can be applied to regenerative medicine aimed at treating rheumatoid arthritis or osteoarthritis, as well as basic research.

### EXAMPLES

The present invention is hereafter described in greater detail with reference to examples, although the technical scope of the present invention is not limited thereto.

### Example 1

### Cartilage tissue engineering from mesenchymal stem cells derived from rabbit bone marrow using collagen sponge in RWV bioreactor

### 1. Experimentation

(1) Preparation of Mesenchymal Stem Cells Derived from Rabbit Bone Marrow

Mesenchymal stem cells derived from rabbit bone marrow were prepared from the femur of a 2-week-old JW rabbit (female) in accordance with the method of Maniatopoulos et al. (Maniatopoulos, C., Sodek, J., and Melcher, A. H., 1988, Cell Tissue Res., 254, pp. 317-330). The harvested cells were cultured in DMEM containing 10% FBS (Sigma) and Antibiotic-Antimycotic (GIBCO BRL) for 3 weeks, and they were allowed to grow.

(2) Culture of Mesenchymal Stem Cells Derived from Rabbit Bone Marrow

The mesenchymal stem cells derived from rabbit bone marrow thus prepared were seeded on collagen sponges (prepared by extracting and purifying type I collagen from the porcine skin, lyophilizing the same, and crosslinking the same) at a density of 1.5×10⁸ cells/cm³ and suspended in 10 ml of DMEM culture medium (Sigma) containing 10⁻⁷M dexamethasone (Sigma), 10 ng/ml TGF-β (Sigma), 50 μg/ml ascorbic acid (Wako), ITS+Premix (BD), 40 μg/ml L-pro line (Sigma), and Antibiotic-Antimycotic (GIBCO BRL). The composite was subjected to rotation culture using an RWV bioreactor (Synthecon) for 3 weeks. As a control example, the cells were directly seeded in the aforementioned solution at the same density and then subjected to rotation culture in an RWV bioreactor in the same manner, without using collagen sponges.

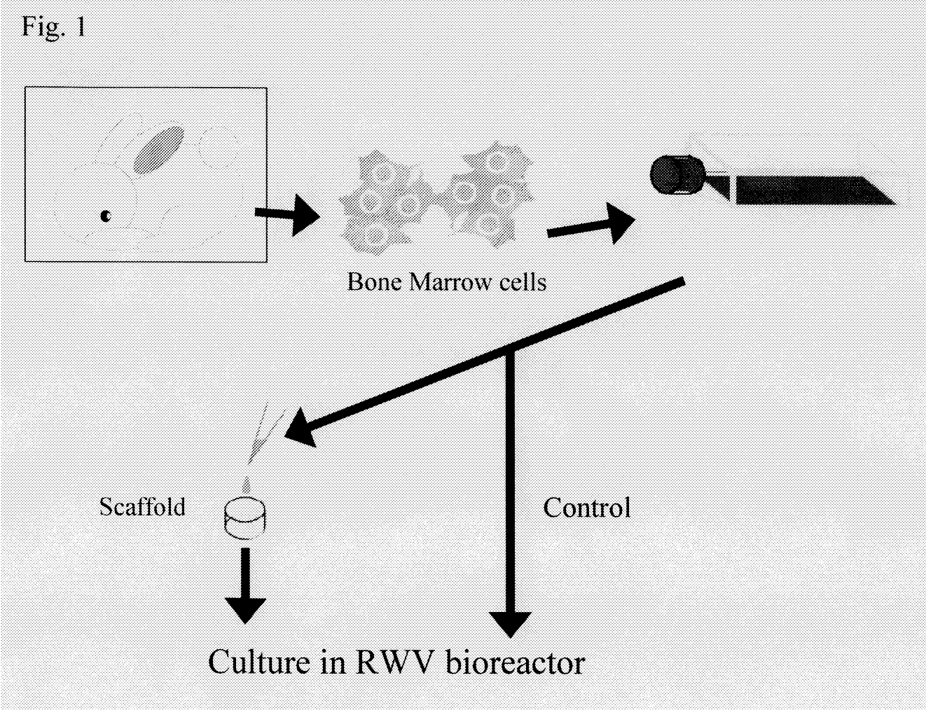

Rotation culture using an RWV bioreactor was carried out using a 5-cm vessel at a rotation speed of 8.0 to 24 rpm at 37° C. in the presence of 5% CO₂. The rotation speed was frequently adjusted manually by visually inspecting the tissue aggregate to maintain a stationary position in the vessel. During culture, bubbles would occur because of cellular respiration, and it would disturb the simulated microgravity environment. Thus, bubbles were frequently removed. FIG. 1 shows the protocol of the present example.

### 2. Evaluation

### (1) Histological Staining

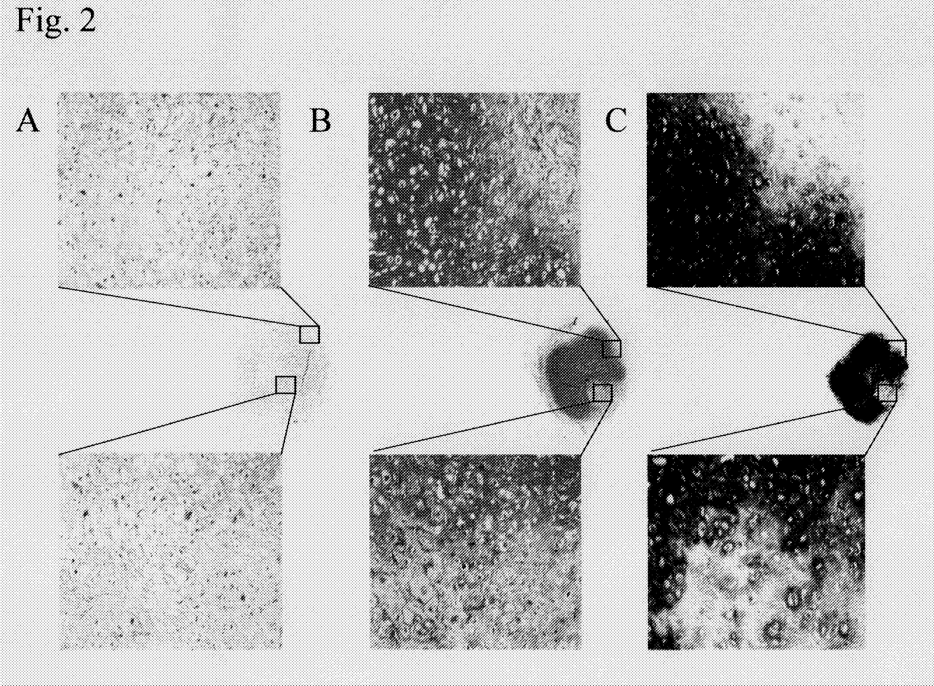

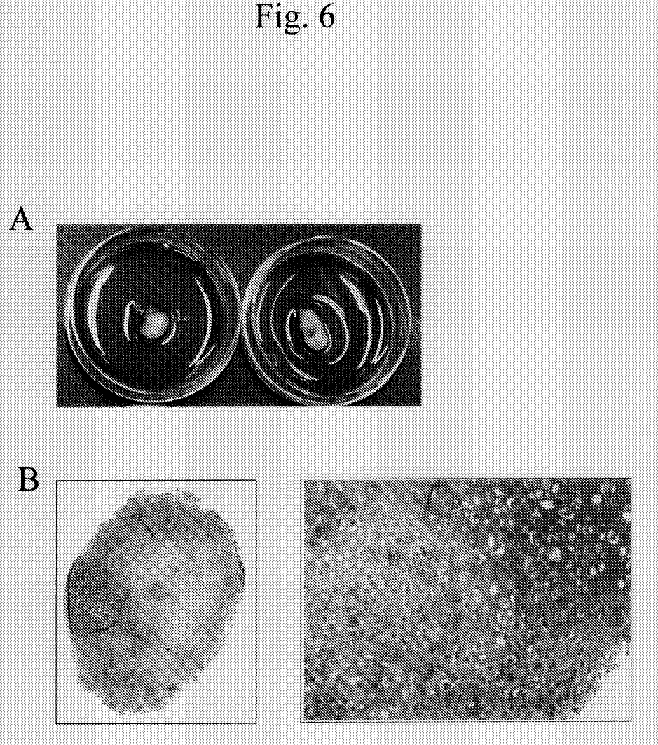

RWV-cultured cartilage tissues obtained with the use of collagen sponges and without the use of collagen sponges (a control) were histologically stained with hematoxylin-eosin (HE), safranin O, and toluidine blue 2 weeks after the initiation of culture to evaluate the capacity for cartilaginous matrix formation. The cultured tissues were fixed with 4% paraformaldehyde and 0.1% glutaraldehyde by microwave radiation. On the next day, the resultants were subjected to decalcification in 10% EDTA and 100 mM Tris (pH 7.4), and decalcification was continued for about 1 week. After the decalcification, the resultants were dehydrated in ethanol and then embedded in paraffin. Sections with a thickness of 5 μm each were prepared. Those sections were then deparaffinized, stained with hematoxylin-eosin, safranin O, and alcian blue in accordance with a conventional technique, and then observed. The results are shown in FIG. 2 and in FIG. 6.

### (2) Assay of Glycosaminoglycan (GAG) Content

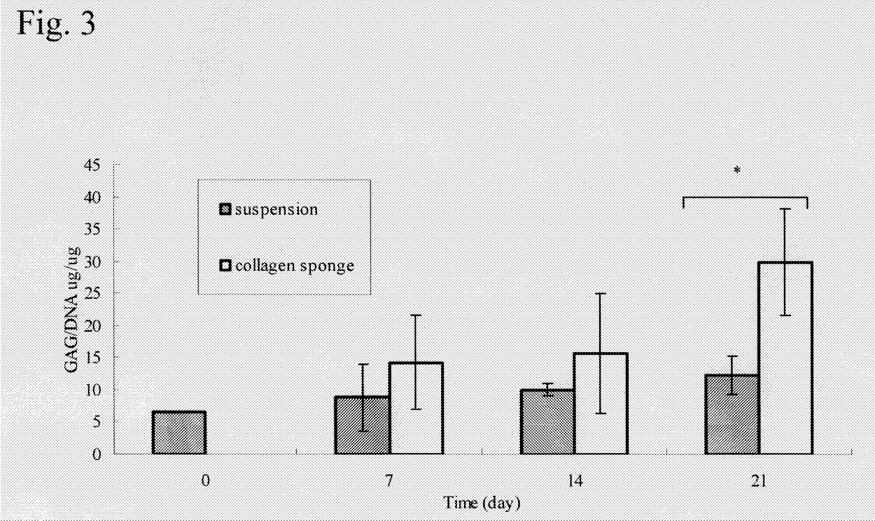

The GAG contents in the RWV-cultured cartilage tissues obtained with the use of collagen sponges and without the use of collagen sponges (a control) were measured every week after the initiation of culture. Assay was carried out by chromoscopy using the Blyscan Glycosaminoglycan Assay kit (Biocolor Ltd.). The results are shown in FIG. 3.

### (3) Compression Strength

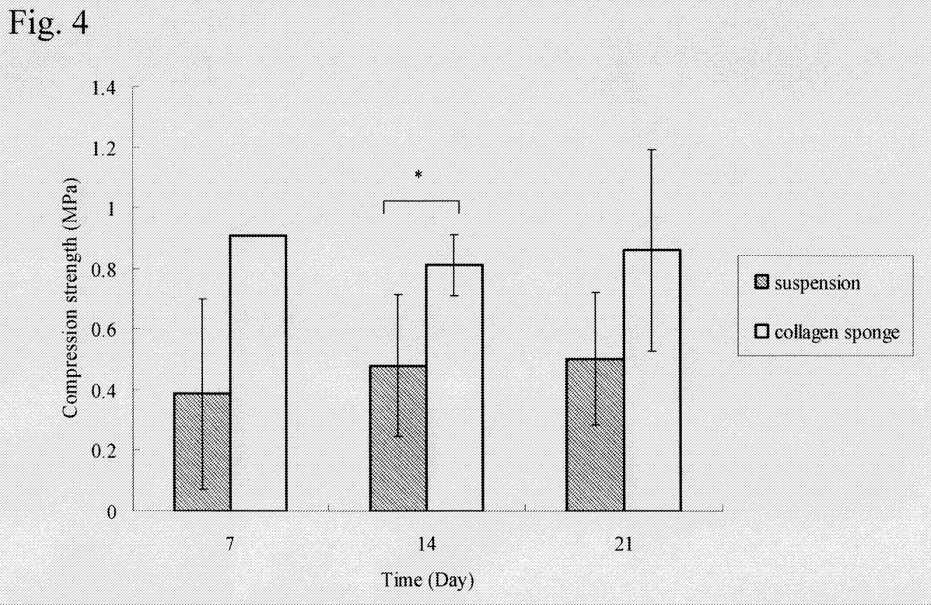

Strength of the RWV-cultured cartilage tissues obtained with the use of collagen sponges and without the use of collagen sponges (a control) were measured using the EIKO TA-XT2i (Eko Instruments). The RWV-cultured cartilage tissue was cut into 2-mm square pieces, such tissue pieces were then compressed at a rate of 0.1 mm/sec, the stress-strain curve was determined from the compression load (Pa) and the distance (mm), and the compression strength was calculated based thereon. The results are shown in FIG. 4.

### (4) Immunostaining

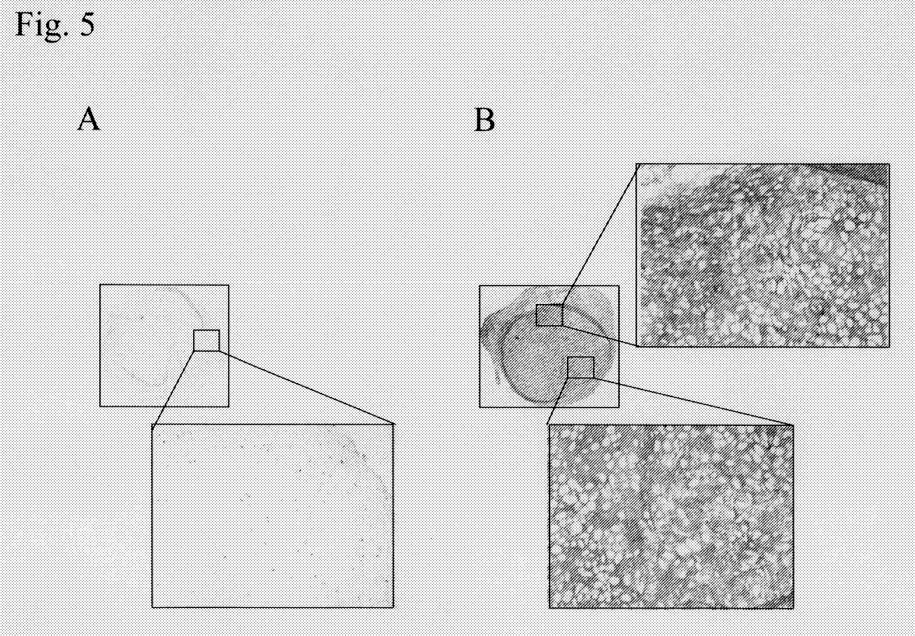

The RWV-cultured cartilage tissue obtained with the use of collagen sponges for 2 or 4 weeks and the RWV-cultured cartilage tissue obtained in the same manner without the use of collagen sponges were subjected to immunostaining using an anti-type I collagen monoclonal antibody (Developmental Studies Hybridoma Bank), an anti-type II collagen monoclonal antibody (Daiichi Fine Chemical Co., Ltd.), and an anti-proteoglycan antibody (Chemicon). The results are shown in FIG. 5 and FIGS. 10 to 12.

### 3. Results

### (1) Histological Staining

The results of hematoxylin-eosin staining, safranin O staining, and toluidine blue staining were excellent (after 2 weeks of culture). The cartilage tissue engineered with the use of collagen sponges was found to sufficiently maintain its initial spongy shape, and homogeneous cartilage tissue was regenerated except for the peripheral portions (FIG. 2).

### (2) GAG Content

The GAG content of the cartilage tissue engineered with the use of collagen sponges was significantly higher than that of the control (FIG. 3).

### (3) Compression Strength

The compression strength of the cartilage tissue engineered with the use of collagen sponges became sufficiently high at the shorter period (about 1 week) than that of the control, and such strength was maintained thereafter (FIG. 4).

### (4) Immunostaining

The cartilage tissue engineered via culture in an RWV bioreactor for 2 weeks with the use of collagen sponges was not substantially stained with an anti-type I collagen antibody, but it was strongly stained with an anti-type II collagen antibody. This represents a typical cartilage feature (FIG. 5).

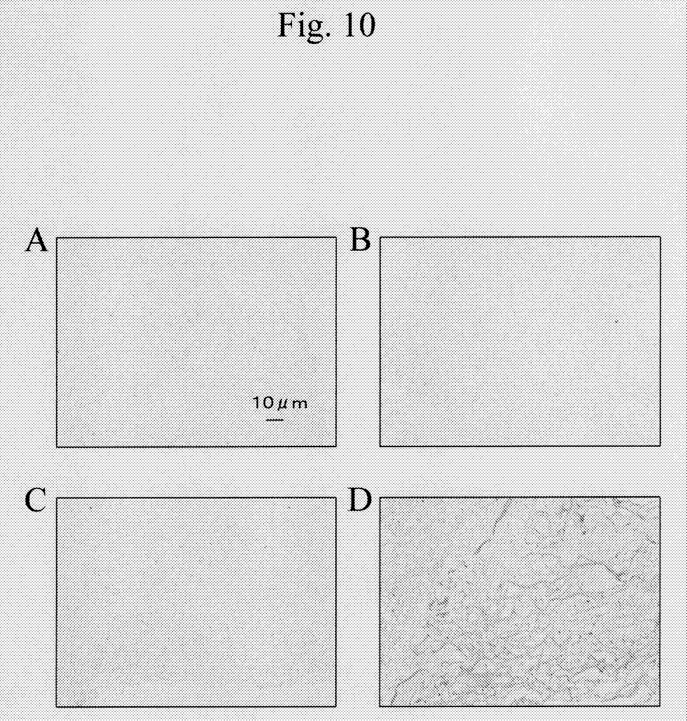

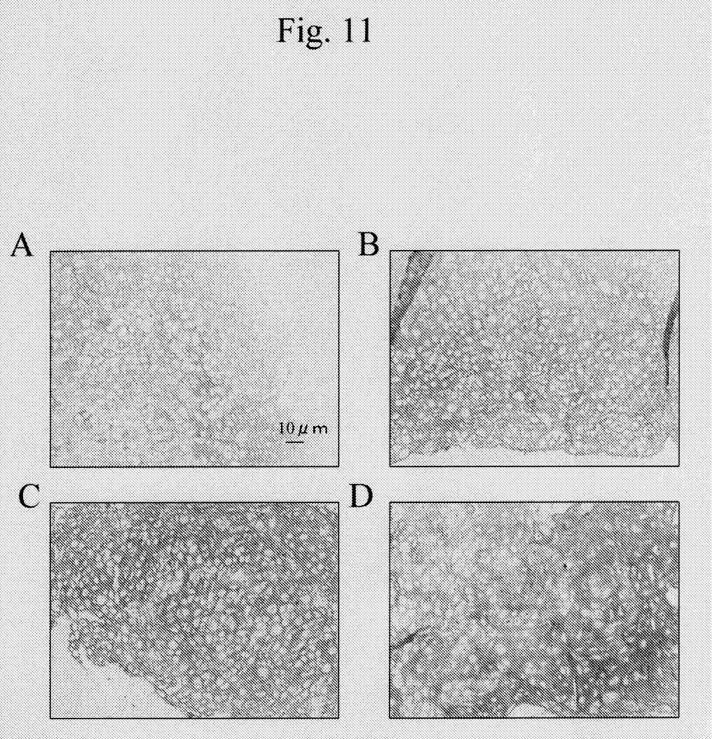

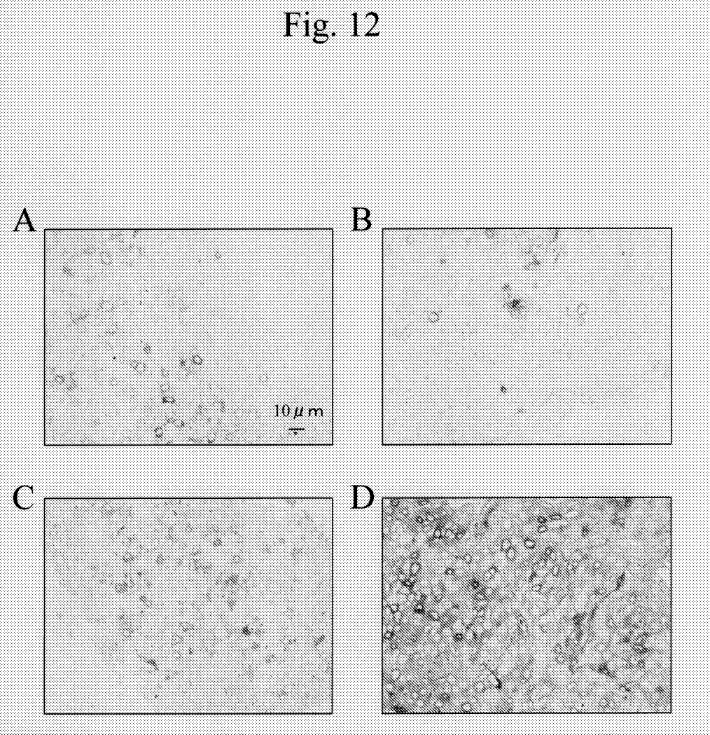

Subsequently, the cartilage tissue engineered with the use of collagen sponges (scaffolds) was compared with that engineered without the use of collagen sponges. The cartilage tissues were not substantially stained via immunostaining with the use of an anti-type I collagen antibody, and the results of staining did not significantly differ, regardless of the culture duration or the use of collagen sponges (FIG. 10). When the cartilage tissues were subjected to immunostaining with the use of an anti-type II collagen antibody, the degree of staining became stronger as the culture period was prolonged from 2 weeks to 4 weeks. Also, when collagen sponges were used, the degree of staining was stronger than that attained without the use of collagen sponges (FIG. 11). Similarly, the degree of staining became stronger via immunostaining with the use of an anti-proteoglycan antibody, as the culture duration was prolonged from 2 weeks to 4 weeks. Also, when collagen sponges were used, the degree of staining was stronger than that attained without the use of collagen sponges (FIG. 12).

Specifically, the expression levels of marker proteins of cartilage, i.e., type II collagen and proteoglycan, were higher when scaffolds (collagen sponges) were used. Thus, use of scaffolds was found to realize more effective cartilage formation.

### (5) Appearance Etc.

Cartilage tissues engineered by seeding cells in a culture medium and culturing the same in an RWV bioreactor for 2 weeks (i.e., the control) varied in shape each time they were cultured (FIG. 6A). Based on the safranin-O-stained image after 2 weeks of culture, cartilaginous matrix secretions were found to vary, and formation of homogeneous cartilage was not observed (especially the center portion) (FIG. 6B).

### 4. Conclusions

Accordingly, RWV rotation culture using collagen sponges as scaffolds was found to be capable of regulation of cartilage tissue configuration and to be capable of engineering of cartilage tissue having excellent cartilaginous matrix and strength. When scaffolds were not used, cells that had been cultured to overconfluence were seeded and a large cartilage tissue mass could then be obtained. When collagen scaffolds were used, however, a cartilage tissue mass could be obtained without culture to overconfluence.

### Example 2

### Comparison of Static Culture and RWV Rotation Culture with the Use of Collagen Sponges

### 1. Experimentation

Bovine articular cartilage was harvested and sliced, and the cartilage matrix was removed with the aid of collagenase and cultured in a common cell culture medium (MEM+10% FBS) to prepare bovine articular cartilage-derived chondrocytes. The bovine articular cartilage-derived chondrocytes were seeded on collagen sponges (prepared by extracting and purifying type I collagen from the porcine skin and lyophilizing the same) at a density of 1.5×10⁸ cells/cm³ and suspended in 10 ml of DMEM culture medium (Sigma) containing 10⁻⁷ M dexamethasone (Sigma), 10 ng/ml TGF-β (Sigma), 50 μg/ml ascorbic acid (Wako), ITS+Premix (BD), 40 μg/ml L-proline (Sigma), and Antibiotic-Antimycotic (GIBCO BRL). The resultant was subjected to static culture (pellet culture) or rotation culture using an RWV bioreactor (Synthecon) for 3 hours.

Static culture was conducted by adding 10 ml of the cell suspension into a 15-ml conical tube, centrifugated at 50 g for 5 minutes to prepare the tissue pellet, and subjecting the tissue pellet to culture at 37° C. in the presence of 5% CO₂. Pellet culture was also performed in the same manner without TGF-β addition. Rotation culture in an RWV bioreactor was carried out in the same manner as in Example 1 using a 5-cm vessel at a rotation speed of 8.0 to 24 rpm at 37° C. in the presence of 5% CO₂.

### 2. Results

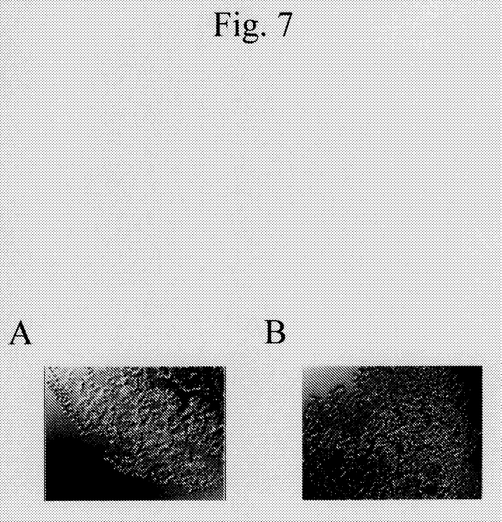

The cells/collagen sponge composites that had been cultured for 2 weeks were nuclear stained by DAPI (Roche). As a result of observation, cells were concentrated on the collagen sponge surface and invasion was not observed in the case of static culture (FIG. 7A). When the collagen sponges were subjected to RWV rotation culture, however, cells were found to be distributed inside the collagen sponges (FIG. 7B).

### Example 3

### Comparison of Various Cellular Scaffolds in Cartilage Tissue Engineering Using RWV Bioreactor

### 1. Experimentation

Using open-cell polylactic acid (OPLA, BD) and a porous composite of hyaluronic acid and hydroxyapatite (hereafter abbreviated as “HAP-HA”) as scaffolds, cartilage tissue regeneration was performed using an RWV bioreactor. OPLA is a synthetic polymer scaffold synthesized from D,D-L,L-polylactic acid (spongy/noncompressive), and the declared pore size thereof is between 100 μm and 200 μm.

Bovine articular chondrocytes that had been prepared in the same manner as in Example 2 were seeded on OPLA and HAP-HA at a density of 1.5×10⁸ cells/cm³ and suspended in 10 ml of DMEM culture medium (Sigma) containing 10⁻⁷ M dexamethasone (Sigma), 10 ng/ml TGF-β (Sigma), 50 μg/ml ascorbic acid (Wako), ITS+Premix (BD), 40 μg/ml L-proline (Sigma), and Antibiotic-Antimycotic (GIBCO BRL). The resultant was subjected to rotation culture using an RWV bioreactor (Synthecon) for 2 weeks.

### 2. Results

### (1) OPLA

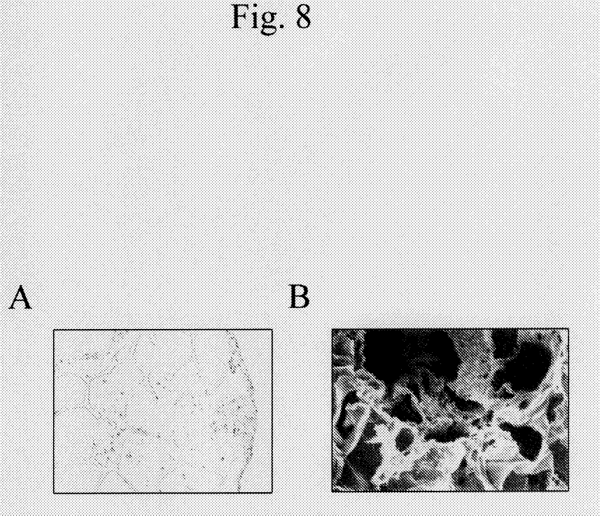

In accordance with the procedure of Example 1, cells/OPLA composite that had been cultured for 2 weeks was stained with toluidine blue, and the stained image was observed. As a result, relatively large numbers of cells were observed around the cell surfaces (FIG. 8A). Further, the similar results were obtained by SEM (scanning electron microscope) observation (SEM-4500, Hitachi).

### (2) HAP-HA

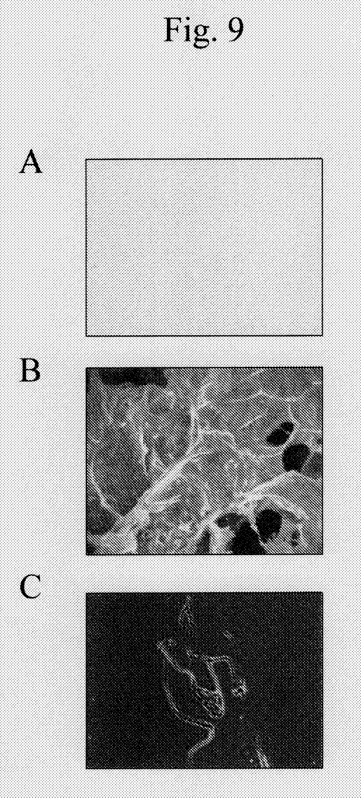

In accordance with the procedure of Example 1, cells/HAP-HA composite that had been cultured for 2 weeks was stained with toluidine blue, and the stained image was observed (FIG. 9A). The image was also observed under a phase contrast microscope (IX70, Olympus) and a scanning electron microscope (SEM-4500, Hitachi) (FIG. 9B and FIG. 9C). All results indicate that cell adhesion to the porous HAP-HA was weaker than that to collagen sponges.

### 3. Conclusions

As described above, OPLA and HAP-HA, which are commonly used as scaffolds, were found to be less suitable for RWV rotation culture than collagen sponges in terms of cell adhesion. As a result, collagen sponges were found to be the most useful scaffolds for RWV rotation culture among the 3 types of tested scaffolds.

When RWV rotation culture is performed, scaffolds are influenced by the flow by rotation. Accordingly, scaffolds are required to have sufficient strength to not be deformed by rotation. Also, scaffolds are required to sufficiently adhere to cells, so that the adhered cells do not remove during rotation. Since collagen has potential to adhere to mesenchymal stem cells, collagen sufficiently adheres to cells, and collagen sponges (and particularly, crosslinked collagen sponges) have high degrees of mechanical strength. Meanwhile, the mechanical strength of OPLA is superior to that of collagen; however, the adhesiveness thereof to cells is inferior to collagen. While hyaluronic acid plays an important role as a matrix in cartilage tissue regeneration, it is not directly involved in adhesion to cells, and HLA-HA used herein does not have very high mechanical strength. Thus, collagen sponges are considered to produce the best results.

At present, polycaprolactone and polyglycolic acid that are used as cellular scaffolds are known to sufficiently adhere to cells and have adequate mechanical strength. Thus, such substances are expected to produce similar effects as collagen sponges.

All publications, patents, and patent applications cited herein are incorporated herein by reference in their entirety.

### INDUSTRIAL APPLICABILTY

According to the present invention, cartilage tissue can be effectively engineered from bone marrow cells without damaging autologous cartilage. The method of the present invention can be applied to regenerative medicine aimed at treating rheumatoid arthritis or osteoarthritis in the orthopedic field or at repairing auricular cartilage in the plastic surgery field, as well as to basic research.

1. A method for cartilage tissue engineering comprising seeding bone marrow cells on scaffolds and culturing the same in a simulated microgravity environment.

2. The method according to claim 1, wherein the scaffolds are collagen-based scaffolds, polymer-based scaffolds such as polylactic acid, polyglycolic acid, polycaprolactone, D,D-L,L-polylactic acid, and hyaluronic acid, or composites thereof.

3. The method according to claim 1, wherein the scaffolds are collagen-based scaffolds or polycaprolactone or polyglycolic acid-based scaffolds.

4. The method according to claim 2, wherein collagen is crosslinked.

5. The method according to claim 1, wherein the simulated microgravity environment provides gravity that is 1/10 to 1/100 of the ground gravity to an object on a time-average basis.

6. The method according to any one of claims 1, wherein the simulated microgravity environment is attained with the use of a uniaxial rotary bioreactor that realizes a simulated microgravity environment on earth by compensating ground gravity by stress resulting from rotation.

7. The method according to claim 6, wherein the uniaxial rotary bioreactor is a rotating wall vessel (RWV) bioreactor.

8. The method according to claim 7, wherein culture is conducted by seeding bone marrow cells at a density of 10⁶ to 10⁷ cells/cm³ at a rotation speed of 8.5 to 25 rpm when a 5-cm RWV vessel is used.

9. The method according to any one of claims 1, wherein culture is conducted by adding TGF-β and/or dexamethasone to a culture medium.

10. The method according to any one of claims 1, wherein the bone marrow cells are harvested from a target in need of cartilage tissue transplantation.

11. The method according to claim 1, wherein the collagen is type I or type II collagen.
